(19) United States
(12) Patent Application Publication
(10) Pub. No.: US 2008/0081071 A1
(43) Pub. Date: Apr. 3, 2008

(54) Film Embedded Packaging and Method of Making Same

(76) Inventors: Pradeep Sanghvi, Schererville, IN (US); Richard C. Fuisz, McLean, VA (US)

Correspondence Address: HOFFMANN & BARON, LLP, 6900 JERICHO TURNPIKE, SYOSSET, NY, 11791, US

(21) Appl. No.: 11/863,420

(22) Filed: Sep. 28, 2007

Related U.S. Application Data
(60) Provisional application No. 60/848,344, filed on Sep. 29, 2006.

(51) Int. Cl. A61K 9/00; B31B 1/64; B65D 85/804

(52) U.S. Cl. 424/484; 206/439; 206/524.7; 493/189

(57) ABSTRACT

The present invention relates to packaging in the form of a pouch, which may contain active substances, such as food products, pharmaceutical agents, nutraceuticals and cosmetic agents, or the like. More specifically, in some embodiments, the present invention provides a pouch, which includes at least one porous substrate encompassing a closed volume and at least one water-soluble film at least partially embedded in the at least one porous substrate. The pouch may contain an active substance within the closed volume, as well as an active substance in the water-soluble film. The present invention also relates to methods of making and using the pouches.

### CROSS REFERENCE TO RELATED APPLICATIONS

This application claims priority to U.S. Provisional Application No. 60/848,344, filed on Sep. 29, 2006, the contents of which are incorporated by reference.

### FIELD OF THE INVENTION

The present invention relates to packaging in the form of a pouch, which may contain active substances, such as food products, pharmaceutical agents, nutraceuticals and cosmetic agents or the like. The pouch material may include a water-soluble film embedded in the pouch which dissolves when the pouch is placed at a selected body site. The present invention also relates to methods of making such pouches, as well as methods of using same.

### BACKGROUND OF THE RELATED TECHNOLOGY

It often is desirable to package drugs, food products and related consumable items into pre-determined amounts. For instance, drug products can be packaged in a porous semi-permeable material. The material is insoluble in water and typically flavorless.

In addition, smokeless tobacco products are conventionally packaged into individual pouches for oral use. Such packaging typically is made from a porous material that is flavorless and insoluble in water. Therefore, the material does not typically dissolve in the mouth during use. The product contained within the pouch, however, flows out through the porous material into the oral cavity during use.

It also is desirable to provide flavors that may be consumed during use of such packaged products. For example, consumers sometimes enjoy experiencing a mint flavor during use of a smokeless tobacco product. Flavorless porous materials, however, have typically been used to form such packages.

Further, undesirable interactions between the packaged product and the porous semi-permeable packaging material sometimes occur in such products Prior known packaging systems have failed to address this problem.

Accordingly, a need exists for improved packaging that avoids the problems encountered in the prior art.

### SUMMARY OF THE INVENTION

In accordance with some embodiments of the present invention, there is provided a pouch for administering an active component, which includes at least one porous semi-permeable substrate encompassing a closed volume; and at least one water-soluble film at least partially embedded in the at least one porous substrate

Some embodiments of the present invention provide a method of making a pouch for administering an active component, which includes the steps of. (a) providing a water-insoluble porous semi-permeable substrate; (b) at least partially embedding the porous substrate with a water-soluble film; and (c) folding the at least partially embedded porous substrate to define a closed volume.

In some embodiments of the present invention, there is provided a method of delivering multiple active components into the body cavity of an individual, which includes the steps of.
(a) providing a pouch including:
   (i) at least one porous semi-permeable substrate encompassing a closed volume;
   (ii) at least one water-soluble film at least partially embedded in the at least one porous substrate, the water-soluble film containing a first active component; and
   (iii) a second active component contained in the closed volume.
(b) applying the pouch into the body cavity of the individual; and
(c) allowing the at least one water-soluble film to dissolve and release the first active component into the body cavity of the individual in combination with the second active component.

Some embodiments of the present invention provide a method of delivering an active component in combination with a tobacco product into the oral cavity of an individual, which includes the steps of:
(a) providing a pouch including:
   (i) at least one porous semi-permeable substrate encompassing a closed volume;
   (ii) at least one water-soluble film at least partially embedded in the at least one porous substrate, the water-soluble film containing an active component; and
   (iii) a tobacco product contained in the closed volume;
(b) applying the pouch into the oral cavity of the individual; and
(c) allowing the at least one water-soluble film to dissolve and release the active component into the oral cavity of the individual in combination with the tobacco product.

The present invention, therefore provides porous substrates used in making packaged products, such as pouches, that are embedded with a water-soluble film. The water-soluble film may contain a flavor that can be experienced along with the edible material housed inside the packaging Alternatively, the water-soluble film may contain a variety of other active substances for use in combination w%ith an active material housed inside the pouch. The pouches of the present invention thereby overcome the shortcomings of the prior art.

### BRIEF DESCRIPTION OF THE DRAWINGS

FIG. 1 is a side elevational view of a pouch in accordance with an embodiment of the present invention;

FIG. 2 is a cross-sectional view taken along line 2-2 of FIGS. 1 and 3; and

FIG. 3 is a cross-sectional view of a pouch in accordance with an embodiment of the present invention.

### DETAILED DESCRIPTION OF THE INVENTION

The present invention relates to packaging in the form of a pouch, which may be administered at a selected body site, such as within the oral cavity. The pouch includes a porous semi-permeable substrate material, which encompasses a closed volume, and a water-soluble film at least partially embedded in the porous substrate.

A material may be contained inside the pouch. Exemplary materials for inclusion inside the pouch include active components, such as food products, pharmaceutical agents, nutraceuticals and cosmetic agents, including flavors, breath fresheners, or the like, but not including tobacco products. Active components also may be incorporated into the water-soluble film used to cover the pouch, such as, for example, flavors or drugs. Upon administration, such as within a body cavity, the water-soluble film dissolves and releases the active contained therein. The active from the film may commingle with the active contained in the pouch as both active components are released into the body cavity.

Alternatively, in some embodiments, the material contained inside the pouch and/or incorporated into the water-soluble film may include tobacco products, such as tobacco, tobacco extracts, synthetic compounds of tobacco, tobacco flavors, or the like. Tobacco products may be used instead of active components in any of the embodiments described herein.

In a preferred embodiment, the pouch is administered in a body cavity. Besides oral administration, a variety of other administration routes are contemplated for the pouches described herein, including but not limited to, buccal, sublingual, transmucosal, periodontal, gingival, nasal, ocular, otic, vaginal, rectal or topical. Buccal is a preferred administration.

The porous semi-permeable substrate may permit moisture, such as saliva or other body fluids, to flow through the pouch, as well as allowing the enclosed active component or a dissolvable extract thereof to flow out of the pouch into the body cavity. The porous substrate also permits an active ingredient in the film to flow into the pouch. The active from the film can then interact with the material inside the pouch, and the material from the pouch and the active from the film can then flow out of the pouch concurrently.

The semi-permeable nature of the substrate means that it allows certain molecular entities to pass through, while holding back others. The level of permeation of the substrate is selective, and can be determined by one skilled in the art. In general, if the pores of the substrate are larger, the substrate will be more permeable. Smaller pores will make the substrate less permeable. The permeability of the substrate can be selected, for example, based upon the nature of the active within the pouch, the rate and manner in which the active dissolves, etc. For example, if the active is a drug, the permeability of the substrate can be selected to provide a particular rate of drug release.

The porous substrate may include a water-insoluble material, such as those materials conventionally used in smokeless tobacco products, tea bags, or the like. Suitable materials include, but are not limited to, fiber, paper, water-insoluble polymers, cloth and fabric. Water-insoluble polymers such as cellulosic polymers may be used. Specific examples of useful water insoluble polymers include, but are not limited to, ethyl cellulose, hydroxypropyl ethyl cellulose, cellulose acetate phthalate, hydroxypropyl methyl cellulose phthalate and combinations thereof. Composite substrates of various materials, such as those mentioned above, also may be used to form the porous substrate.

In some embodiments, the porous substrate may be at least partially embedded with the water-soluble film. The water-soluble film may have any suitable thickness, for example, a thickness of about 20 microns to about 100 microns. The water-soluble film may dissolve when contacted with moisture at the administration site within the body, such as in the oral cavity. The dissolution rate of the water-soluble film may be adjusted for different embodiments to provide different release rates of the active component contained therein. For example, in some embodiments, the water-soluble film may have a rapid dissolution rate, such as about 1-2 minutes, which provides a rapid release of the active. In other embodiments, the water-soluble film may be adapted to have a slower dissolution rate, such as 30-60 minutes or even up to about 24 hours, which sustains the release of the active component contained in the film. A variety of different factors may affect the dissolution rate of the film, including the film-forming polymers selected and film thickness, among others.

The water-soluble film may include at least one water-soluble polymer. As used herein the phrase “water soluble polymer” and variants thereof refer to a polymer that is at least partially soluble in water, and desirably fully or predominantly soluble in water, or absorbs water.

In some embodiments, the water-soluble polymer may be capable of heat-sealing along With the porous substrate to form a sealed pouch. In addition, different water-soluble polymers or combinations of polymers may be used to adjust the dissolution rate of the film. The dissolution rate also may be adjusted by combining water-soluble polymers having different viscosities or molecular weights.

For instance, in some embodiments, the water-soluble polymer may include polyethylene oxide, alone or in combination with other water-soluble polymers. Water-soluble cellulosic polymers, such as hydroxypropyl cellulose and hydroxypropyl methylcellulose may be employed. Hydroxypropyl methylcellulose, in particular, is capable of heat sealing with the porous substrate material without melting to an undesirable degree.

The molecular weight of polyethylene oxide used in the films may range, for example, from about 100,000 to about 5 million. In addition, blends of different molecular weight polyethylene oxides may be employed, as described in Assignee's co-pending U.S. application Ser. No. 10/856,176 (U.S. Patent Publication No. 2005/0037055 A1), filed on May 28, 2004, the contents of which are incorporated herein by reference in their entirety.

In some embodiments, water-soluble polymers, such as cellulosic polymers, having different viscosities may be used. For example, the water-soluble polymer may include a combination of hydroxypropyl methylcellulose having a viscosity of about 15 cps with hydroxypropyl methylcellulose having a viscosity of about 50 cps. The addition of the higher viscosity hydroxypropyl methylcellulose may impart a slower dissolution rate to the film, such as about 30-60 minutes, which may be desirable in some embodiments. Additionally, the higher viscosity hydroxypropyl methylcellulose may act to encapsulate the active component contained in the film to some degree. Such encapsulation may extend the release of the active over even longer periods of time.

Commercially available examples of such polymers include METHOCEL E15 (hydroxypropyl methylcellulose having an apparent viscosity of 15 cps) and METHOCEL E50 (hydroxypropyl methylcellulose having an apparent viscosity of 50 cps), both available from the Dow Chemical Company.

Examples of other suitable water-soluble polymers for use in the water-soluble films include, but are not limited to, pullulan, hydroxyethyl cellulose, polyvinyl pyrrolidone, carboxymethyl cellulose, polyvinyl alcohol, sodium alginate, polyethylene glycol, xanthan gum, tragancanth gum, guar gum, acacia gum, arabic gum, poly acrylic acid, methylmethacrylate copolymer, carboxyvinyl copolymers, starch, gelatin, and combinations thereof The use of such polymers in film are described in detail in U.S. application Ser. No. 10/856,176, referred to above.

In some embodiments, it also may be desirable to add polydextrose to the water-soluble film. Polydextrose is a water-soluble polymer that serves as a filler and solubility enhancer, i.e., it increases the dissolution time of the film, without compromising the sealing properties of the film. Polydextrose may be present in amounts of about 5% to about 30% by weight of film, more specifically 9% to about 15% by weight.

A variety of optional additives also may be included in the water-soluble film, such as, but not limited to, anti-foaming agents, such as silicone-containing compounds, anti-tacking agents, plasticizers, polyalcohols, surractants and thermo-setting gels such as pectin, carageenan, and gelatin among others.

Processes for preparing water-soluble films are described in assignee's co-pending U.S. patent application Ser. No. 10/074,272, filed on Feb. 14, 2002, and published as U.S. Patent Publication No. 2003/0107149 A1, the contents of which are incorporated herein by reference in their entirety.

In some embodiments, the water-soluble film itself also malt include at least one active component. At least one active component, such as food products, pharmaceutical agents, nutraceuticals or cosmetic agents, also may be contained in the closed volume of the pouch. The active component contained in the water-soluble film may be the same or different from the active housed in the pouch.

In some embodiments, suitable actives for housing in the pouch and/or for incorporation into the water-soluble film include, but are not limited to: food products; botanicals; herbals; minerals; insects; nutraceuticals; pharmaceutical agents; cosmetic agents; drugs; bioactive active substances; medicaments, antidotes; vaccines; antigens or allergens; mouthwash components; flavors; fragrances; enzymes; preservatives; sweetening agents; colorants; spices, vitamins; polyphenols; phytochemicals; and combinations thereof. Such actives do not include tobacco products.

Examples of botanicals include, without limitation: roots; barks; leaves; stems; flowers; fruits: sunflower seeds, and combinations thereof.

Examples of herbals include, without limitation: agrimony, alfalfa, aloe, angelica, anise, arjuna, amica, Artemisia, ashwagandha, astragalus, avena, barberry, bayberry bilberry, bdellium gum, bilberry, birch, bissy nut, bitter orange, black cohosh, black currant oil, black walnut, blessed thistle, blue cohosh, blue vervain, borage, burdock, burdock, butcher's broom, calendula, cascara sagrada, catnip, cat's claw, cayenne, celery seed, chamomile, chaparral, chickweed, chrysanthemum, cinnamon, cleavers, clove, comfrey, coptis, cordyceps, cranberry, cyani flowers, damiana, dan shen, dandelion, devil's claw, dong quai, Echinacea, elderberry, elecampane, ephedra, eucalyptus, eyebright, false unicorn, fennel seed, fenugreek, feverfew, flax seed oil, garcinia cambogia, garlic, gentian, ginger, gingko, ginseng, goldenseal, gotu kola, hawthorn, holy basil, ho she wu, hops, horehound root, horseradish, horsetail, hydrangea, hyssop, irish moss, juniper berry, kava, kelp, khella, lady slipper, lamb's quarter, lavender, lemon balm, licorice, lobelia, male fern, mandrake, marshmallow root, mate, medical marijuana, milk thistle, morinda, motherwort, mullein, myrrh, nasturtium, neem oil, noni, oatstraw, olive leaf, oregano oil, Oregon grape root, pan pien pien, papaya, parruva brava, parsley, passionflower, pau d'arco, peppermint, periwinkle, pippali fruit, poke, prickly ash, psyllium, queen of the meadow, quercetin, raspberry leaf, red clover, reishi, rhubarb, rooibos, rosemary, safflower, sage, sarsaparilla, saw palmetto, schisandra, senega root, senna, skullcap, sheep sorrel, shepherd's purse, shiitake, Siberian ginseng, slippery elm, spirulina, squaw vine, St. John's wort, stinging nettle, suma, tea tree oil, thyme, turkey rhubarb, turmeric, usnea, uva ursi, valerian, vitex, watermelon seeds, white oak, white willow, wild cherry bark, wild yam, willow bark, wood betony, wormwood, yarrow, yellow dock, yerba santa; and combinations thereof.

A wide variety of medicaments, bioactive active substances and pharmaceutical agents may be included as an active in the water-soluble film. Examples of useful drugs include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-park-insonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids such as bromocryptine and nicotine, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Examples of medicating active ingredients include antacids, H₂-antagonists, and analgesics. For example, antacid dosages can be prepared using the ingredients calcium carbonate alone or in combination with magnesium hydroxide, and/or aluminum hydroxide. Moreover, antacids can be used in combination with H₂-antagonists.

Analgesics include opiates and opiate derivatives, such as oxycodone (available as Oxycontin®), ibuprofen, aspirin, acetaminophen, and combinations thereof that may optionally include caffeine.

Other drugs include anti-diarrheals such as immodium AD, anti-histamines, anti-tussives, decongestants, vitamins, and breath fresheners. Suitable vitamins contemplated for use herein include any conventionally known vitamins, such as, but not limited to, Vitamins A, B, C and E. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the film compositions of the present invention.

Also contemplated for use herein are anxiolytics such as alprazolam (available as Xanax®), anti-psychotics such as clozopin (available as Clozaril®) and haloperidol (available as Haldol®); non-steroidal anti-inflammatories (NSAID's) such as dicyclofenacs (available as Voltaren®) and etodolac (available as Lodine®), anti-histamines such as loratadine (available as Claritin®), astemiizole (available as Hismanal™), nabumetone (available as Relafen®), and Clemastine (available as Tavist®); anti-emetics such as granisetron hydrochloride (available as Kytril®) and nabilone (available as Cesamet™); bronchodilators such as Bentolin®, albuterol sulfate (available as Proventil®); anti-depressants such as fluoxetine hydrochloride (available as Prozac®), sertraline hydrochloride (available as Zoloft®), and paroxtine hydrochloride (available as Paxil®); anti-migraines such as Imigra®, ACE-inhibitors such as enalaprilat (available as Vasotec®), captopril (available as Capoten®) and lisinopril (available as Zestril®); anti-Alzheimer's agents, such as nicergoline; and Ca^{H}-antagonists such as nifedipine (available as Procardia® and Adalat®), and verapamil hydrochloride (available as Calan®).

Erectile dysfunction therapies include, but are not limited to, drugs for facilitating blood flow to the penis, and for effecting autonomic nervous activities, such as increasing parasympathetic (cholinergic) and decreasing sympathetic (adrenersic) activities. Useful non-limiting drugs include sildenafils such as Viagra®, tadalafils, such as Cialis®, vardenafils, apomorphines, such as Uprima®, yohimbine hydrochlorides such as Aphrodyne®, and alprostadils such as Caverject®.

The popular H₂-antagonists that are contemplated for use herein include, but are not limited to, cimetidine, ranitidine hydrochloride, famotidine, nizatidien, ebrotidine, mifentidine, roxatidine, pisatidine and aceroxatidine.

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

The pharmaceutically active agents may include allergens or antigens, such as but not limited to, plant pollens from grasses, trees, or ragweed; animal danders, which are tiny scales shed from the skin and hair of cats and other furred animals; insects, such as house dust mites, bees, and wasps; and drugs, such as penicillin.

An anti-oxidant also may be added to prevent the degradation of an active, especially where the active is photosensitive.

The bioactive active substances employed herein may include beneficial bacteria. More specifically, certain bacteria normally exist on the surface of the tongue and in the back of the throat. Such bacteria assist in the digestion of food by breaking down proteins found in the food. It may be desirable, therefore, to incorporate these bacteria into some embodiments of the present invention.

It also may be desirable to include actives for treating breath malodor and related oral care conditions, such as actives which are effective in suppressing microorganisms. Because breath malodor can be caused by the presence or anaerobic bacteria in the oral cavity, which generate volatile sulfur compounds, components that suppress such microorganisms may be desirable. Examples of such components include antimicrobials such as triclosan, chlorine dioxide, chlorates, and chlorites, among others. The use of chlorites, particularly sodium chlorite, in oral care compositions such as mouthrinses and toothpastes is taught in U.S. Pat. Nos. 6,251,372, 6,132,702, 6,077,502, and 6,696,047, all of which are incorporated herein by reference. Such components are incorporated in amounts effective to treat malodor and related oral conditions.

Cosmetic active agents may include breath freshening compounds like menthol, other flavors or fragrances, especially those used for oral hygiene, as well as actives used in dental and oral cleansing such as quaternary ammonium bases. The effect of flavors may be enhanced using flavor enhancers like tartaric acid, citric acid, vanillin, or the like.

Examples of polyphenols include, without limitation, flavonoids, such as catechins, epicatechins, procyaidins and anthrocyanins, among others.

Examples of phytochemicals include, without limitation, allyl sulfides, indoles, glucosinolates, sulfaforaphane, isothiocyanates, thiocyanates, thiols, lycopene, carotenoids, phthalides, polyacetylenes, silymarin, monoterpenes, ellagic acid, phenols, flavonoids, phytic acid, saponins, gingerols and glycyrrhizin catechins, among others.

Also color additives may be employed. In some embodiments, it may be desirable to add colorants to the water-soluble film to enhance the overall aesthetic appearance of the pouch. For instance, the active component housed within the pouch may cause undesirable staining of the porous substrates forming the pouch. The film may include a colorant or whitening agent that masks such undesirable staining, thereby improving the appearance of the pouch. Such color additives include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide.

Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors.

The flavorings may be added to provide a hot or cold flavored drink or soup. These flavorings include, without limitation, tea and soup flavorings such as beef and chicken.

Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus Fruits), tolyl aldehyde (cherry, almond), 2,6-dimethyloctanol (green fruit), and 2-dodecenal (citrus, mandarin), combinations thereof and the like.

Flavors may be present in the water-soluble film in amounts of about 5% to about 30% by weight of the film, more specifically about 15% to about 27% by weight of the film.

Alternatively, in some embodiments, the material housed in the pouch and/or incorporated into the water-soluble film may include one or more tobacco products, such as smokeless tobacco, tobacco extracts, synthetic compounds of tobacco, tobacco flavors, snuff, or the like. Tobacco products also may be used in combination with any of the active components described herein.

Some embodiments also may include an emulsification system in the water-soluble film. An emulsification system may be used to alleviate non-uniform patterns created in the film by flavors, particularly in embodiments incorporating high levels of flavor, such as about 25-30% by weight of thie film composition, for an intense flavor impact. Non-uniform patterns may create an adverse film appearance, and thus, may be undesirable in some embodiments. The emulsification system may include any of a variety of emulsifiers, such as, for example, propylene glycol alginate, polyoxyethylene sorbitan monooleate (Polysorbate 80) and/or sorbitan monooleate. In some embodiments, the emulsification system may include propylene glycol alginate in amounts of about 0.5% to about 1.5% by weight of the film, polyoxyethylene sorbitanl monooleate in amounts of about 0.1% to about 1% by weight of the film and sorbitan monooleate in amounts of about 0.1% to about 1% by weight of the film.

Actives in the water-soluble film also may include sweetening agents. The sweeteners may be chosen from the followving non-limiting list: glucose (corn svrup), dextrose, invert sugar, fructose, and combinations thereof, saccharin and its various salts such as the sodium salt, dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

In general, the active components contained in the water-soluble film may be present in amounts of about 0.001% to about 50% by weight of the film, more specifically about 1% to about 27% by weight of the film.

In some embodiments, the wvater-soluble film may include an ionic component to impart or maintain a charged environment to the film. In particular, imparting or maintaining an ionic charge on the surface of the film lining or cover may affect the adhesion properties of the film to the mucosal surfaces. Any component that can impart a net (+) or (−) ionic charge may be used. For instance, acids, bases, salts or any polymers that are capable of imparting an ionic charge may be included in the water-soluble film.

Any of the active components described above may be incorporated into the water-soluble Film and/or housed in the closed volume of the pouch. In some embodiments, a different active component may be contained in the pouch from the active component incorporated into the water-soluble film. For example, a flavor may be incorporated into the film and a food product contained in the pouch. Alternatively, some embodiments may include the same active component in the water-soluble film and within the pouch. Additionally, multiple active components may be incorporated into the water-soluble film and/or contained in the pouch.

Suitable active components and details of water-soluble film formation are more fully described in assignee's co-pending U.S. application Ser. Nos. 10/074,272 and 10/8564176, referred to above, as well as assignee's co-pending U.S. application Ser. No. 10/768,809, filed on Jan. 30, 2004, the contents of which are incorporated herein by reference in their entirety.

As mentioned above, the water-soluble film may be at least partially embedded in the porous substrate. In some embodiments, the water-soluble film may be wholly embedded in the porous substrate. The at least partially film-embedded porous substrate may be formed into a pouch in a variety of different manners.

When forming the film-embedded porous substrate, it is preferred that a substrate with a suitable tensile strength and porosity be chosen. These factors can be determined by one skilled in the art. For example, if the film embedded substrate is to be used for a pouch to be administered orally, the substrate should have a sufficient tensile strength so as to not tear while in the user's mouth. In addition, the substrate should have sufficient porosity to permit the desired release of the active Within the pouch and/or within the film. In addition, the substrate should have sufficient porosity to permit the film-forming solution to be absorbed by the substrate when manufacturing the film-embedded substrate. The porosity patterns of a substrate can be defined to allow for specific flow of the film-forming solution through the substrate.

Once the substrate is chosen, a film-forming solution is applied to the substrate. When the film-forming solution is applied to the substrate, it is preferred that the substrate remain flat and without creases. The film-forming solution is preferably applied to the substrate instead of the substrate being applied to the solution. In this way, the appropriate amount of solution can be applied to the substrate. It is preferred that the solution be applied in an amount sufficient to fully saturate the substrate. In other words, it is preferred that the empty space within the pores of the porous substrate be filled with the film-forming solution. In another embodiment, an amount of film-forming solution is applied to only partially embed the substrate with the water-soluble film.

When applying the film-forming solution to the substrate, it is preferred that the substrate be placed on a supporting material, for example a layer of high density polyethylene (HDPE), polyethylene terepthalate (PET), or paper. The supporting layer prevents the film-forming solution from dripping through the filter paper and allows the flow of the solution between the filter paper and the supporting layer, thus embedding the filter paper with the film. In addition, the supporting later could have adhesive properties to allow attachment to skin or mucosal layer.

In another embodiment, the film is cast on a steel or a metal band or sheet. This method is therefore called bandcasting. In this method, instead of an inert supportive substrate, a machine is equipped with a long steel band on which the film is cast, thus supporting the film. Once the film is dried, the film is taken off the band and packaged, thus avoiding the extra use or cost of the supportive substrate, such as PET. The steel band can be cleaned and reused again. Since, in this case, the film is embedded in a porous semi-permeable substrate, the substrate itself has enough strength to support the film and therefore can be bandcasted.

In a preferred embodiment, the substrate is wetted before the application of the film-forming solution and before attachment to the supporting material. The substrate can be wetted with a liquid suitable for use in a human. For example, the substrate can be wetted with water or 0.5% Tween solution. Such wetting allows the film-forming solution to more uniformly flow through the substrate. Wetting also prevents formation of air pockets in the film-embedded structure. Wetting the substrate also helps secure the substrate to the supporting material when used.

The substrate is then dried such that the film-forming solution forms a film that is at least partially embedded in the porous substrate. The film-embedded substrate can then be used for further processing, such as the formation of a pouch. A film-embedded substrate can be slit into desired width while attached to the supporting layer or can be removed from the supporting layer prior to slitting.

During processing, the porous substrate can be simultaneously fed into a film coating/casting machine along with the supporting layer, with the supporting layer underneath. Proper tension should be applied on the rollers through which the film-forming solution is fed into the coating machine, to avoid formation of creases. This will allow film-forming solution to uniformly coat the substrate surface, seep into the substrate and flow between the substrate and the supporting layer, thus coating the underneath surface of the substrate. The solution is then dried, embedding the film in the substrate.

Another method for processing includes laminating the substrate on the supporting layer by heat, static or other physical or chemical methods. The combined laminated layers can then be fed through the coating machine where the substrate is coated with the film-forming solution and dried.

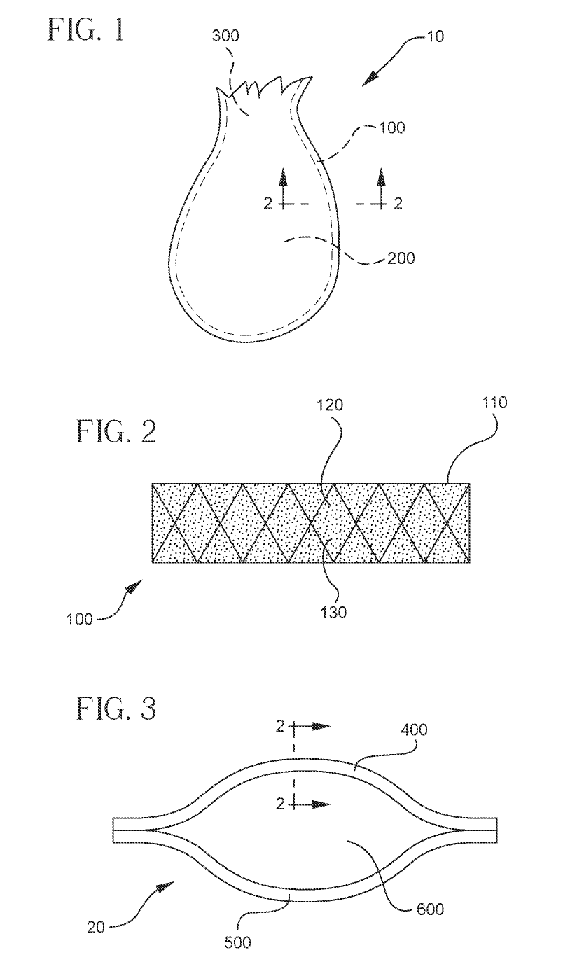

In some embodiments, the film-embedded substrate may be folded such that a closed volume is defined to form a pouch. For example, as shown in FIG. 1, the film embedded substrate may be folded and gathered into a pouch 10 having pouch wall 100 and enclosing volume 200. The film-embedded substrate may be sealed to itself, such as heat sealed, at the gathering point 300 of the pouch 10.

As shown in FIG. 2, taken along the 2-2 axis of FIG. 1, the pouch wall 100 may include a porous substrate 110. The porous substrate includes pores 120 that are embedded with the water-soluble film 130.

In an alternative embodiment, two porous substrates may be provided. The two porous substrates may be sheet-like members. As shown in FIG. 3, two porous substrates may be in perimetric face-to-face engagement with one another defining wall 400 and wall 500 of pouch 20 and enclosing volume 600. The porous substrates may be fused to one another at the perimetric face-to-face engagement. Each substrate defining wall 400 and wall 500 of pouch 20 include pores 120 that are embedded with the water-soluble film 130 as shown in the 2-2 axis in FIG. 2.

A variety of other manners of folding a single porous substrate or multiple porous substrates into a pouch may be employed. For example, a single porous substrate may be folded over itself into a tube-like shape. The tube-like porous substrate may be sealed along its length and at each end to define a closed volume within. The inner and/or outer surfaces of the tube-like porous substrate may be at least partially embedded with a water-soluble film. Other manners of folding and sealing the porous substrate(s) are considered well within the scope of the present invention.

The present invention also is directed to methods of making the pouches described above. In accordance therewith, a water-insoluble porous substrate may be provided. The porous substrate may be at least partially embedded with a water-soluble film. Once the porous substrate has been embedded with the water-soluble film, it may be folded to define a closed volume, thereby forming a pouch.

In some embodiments, the film-embedded porous substrate may be gathered or folded over itself and heat-sealed to itself at the points of contact. For example, in some embodiments, a film-embedded porous substrate may be folded over itself such that one portion of the substrate is engaged along the perimeter with a second portion of the substrate. The substrate may be heat-sealed at the perimetric points of engagement.

In other embodiments, for example, two film-embedded porous substrates, which are in perimetric face-to-face engagement, may be fused or heat-sealed to one another along at least a portion of the perimetric face-to-face engagement. In some embodiments, the water-soluble film may be heat-sealed with the porous substrate.

Prior to heat sealing the pouch, an active component may be positioned within the closed volume defined therein. Any of the active components described above may be housed in the pouch. Alternatively, material can be added to the closed volume after the closed volume is formed.

In some embodiments, for example, the at least partially film-covered porous substrate may be folded over itself to form a pouch having a closed volume. Two sides of tile pouch may be sealed closed, leaving one side of the pouch open. An active component or a tobacco product may be filled into the closed volume via the open side of the pouch. The open side of the pouch then may be sealed closed to form the final product. For instance, the sides of the pouch may be sealed by heat and/or pressure.

Alternatively, in some embodiments, a strand of pouches may be formed in which one side of the strand of pouches is open. A portion of an active component or a tobacco product may be filled into each pouch. Subsequently, the open side of the strand of pouches may be sealed closed and individual pouches may be produced by severing them from the strand. This process is described in more detail in U.S. Pat. No. 5,174,088 to Focke et al., which is incorporated herein by reference in its entirety.

The present invention also is directed to methods of delivering multiple active components into the oral cavity of an individual. In accordance with such methods, a pouch may be provided. The pouch may include at least one porous substrate encompassing a closed volume. In addition, at least one water-soluble film may be at least partially embedded in the porous substrate. The water-soluble film may include a first active component. The water-soluble film also may include any of the other components described above. A second active component may be contained in the closed volume of the pouch. The first and second active components may be the same or different. The pouch then may be applied into a body cavity of an individual. For example, if applied into the oral cavity, as saliva begins to mix with the pouch, the water-soluble film may be allowed to dissolve and release the first active component into the oral cavity of the individual. Desirably, the second active component may release from the pouch into the oral cavity as ell, in combination with the first active component.

More specifically, in some embodiments, as the first active component releases from the water-soluble film, it may combine with the second active component housed in the pouch. A portion of the first active component may be sorbed by the second active component as it is released from the water-soluble film. The sorbed concentration of the first active component may increase as more film dissolves. Then, as saliva mixes with the pouch and reaches the enclosed second active component, a portion of the first active sorbed in the second active also may mix with the saliva and release from the pouch. Such mechanism may provide an extended release of the first active component into the oral cavity of the individual. For instance, if the first active component is a flavor, this mechanism may provide an extended flavor release throughout the product use. Moreover, the sorption of the first active component may be manipulated by varying the moisture content of the second active component housed in the pouch.

Alternatively, methods are provided for delivering an active component in combination with a tobacco product into the oral cavity of an individual. Similar to above, a pouch may be provided. The pouch may include at least one porous substrate encompassing a closed volume. In addition, at least one water-soluble film may be at least partially embedded in the porous substrate. The water-soluble film may include an active component. The water-soluble film also may include any of the other components described above. A tobacco product may be contained in the closed volume of the pouch. The pouch may be applied into the oral cavity of an individual. Once applied into the oral cavity, and as saliva begins to mix with the pouch, the water-soluble film may be allowed to dissolve and release the active component into the tobacco product and into the oral cavity of the individual. Desirably, the tobacco product may release from the pouch into the oral cavity as well, in combination with the active component.

For example, in one embodiment, the active in the film may be a flavoring agent, such as a mint flavoring agent, and the material in the pouch can be tobacco. When saliva contacts the film embedded pouch, the film begins to dissolve and release the flavor agent. This flavor agent travels in two directions. First, the flavor from the film travels out into the mouth cavity. In addition, the flavor from the film travels inward to the tobacco mass inside the pouch where it interacts with the spongy tobacco mass. As the tobacco mass is chewed or squeezed between the consumer's cheek and gum, mint flavored tobacco juice is forced out of the porous substrate pouch.

### EXAMPLES

### Example 1

Film-embedded pouches of the present invention were prepared in accordance with the following. Water-soluble film-forming solutions for use in embedding the film in the porous substrates of the pouches were prepared using the amounts described in Table 1.

TABLE 1

| Component | Weight % |
|---|---|
| Hydroxypropyl methylcellulose (15 cps) | 35.00 |
| Hydroxypropyl methylcellulose (50 cps) | 8.69 |
| Polyethylene oxide | 8.15 |
| Polydextrose | 11.14 |
| Propylene glycol alginate¹ | 1.00 |
| Glycerol monooleate² | 1.00 |
| Polysorbate 80³ | 0.30 |
| Sorbitan monooleate⁴ | 0.20 |
| Propylene glycol | 5.00 |
| Glycerin | 5.00 |
| Amorphous precipitated silica⁵ | 1.00 |
| Magnesium stearate | 0.50 |
| Methyl paraben | 0.02 |
| Sucralose | 2.00 |
| Flavor | 20.00 |
| Hydrophilic titanium dioxide | 1.00 |

¹Commercially available as Colloid 602
²Commercially available as ALDO MO
³Commercially available as T SOL P-80
⁴Commercially available as Crill 4 NF
⁵Commercially available as Sipernat from Degussa (or SAPS FK500LS)

Water was added to a beaker with the glycerol monooleate, Polysorbate 80, sorbitan monooleate, propylene glycol and glycerin. The beaker was secured on a hot plate with a clamp. Agitation was initiated with a mixing blade of a mixer apparatus and the propylene glycol alginate, titanium dioxide and methyl paraben were slurried into the batch. Mixing continued for 10 minutes. The batch was heated to 85° C. and then the hydroxypropyl methylcellulose (15 cps) was slurried in, followed by the hydroxypropyl methylcellulose (50 cps). The batch was mixed until dispersed evenly. The polyethylene oxide was slurried into the batch and mixed until dispersed evenly. The polydextrose and sucralose were slurried into the batch and mixed until dispersed evenly. Agitation was ceased and the silica and magnesium stearate were added to the batch. Agitation was initiated again at a low speed (setting 1). Mixing continued for 5 minutes and then the batch was removed from the heat. As the solution began to gain viscosity (thicken), the agitation speed was slowly lowered to allow the mix to cool quicker. Once the solution reached room temperature, it was mixed on first gear (setting 3). Mixing continued until the polymers were hydrated. The solution was removed from the mixer and split into four 200 gram batches.

A different flavor combination was added to each of the four batches. Three different batches of a brown sugar and cinnamon flavor were used. The fourth batch was a brown sugar and vanilla flavor. Flavor agents were added to the four batches as set forth in Table 2 below:

TABLE 2

| 200 gram batch (E15 = 14.00) | Wt % of Film Composition | Grams |
|---|---|---|
| Batch #1 | | |
| Brown #3 9673-50-2 | 18.00% | 7.20 g |
| Cinnamon 656860 | 7.00% | 2.80 g |
| Batch #2 | | |
| Brown #3 9673-50-2 | 20.00% | 8.00 g |
| Cinnamon 656858 | 4.00% | 1.60 g |
| Batch #3 | | |
| Brown #3 9673-50-2 | 20.00% | 8.00 g |
| Ground Cinnamon FN4517 | 4.00% | 1.60 g |
| Batch #4 | | |
| Brown #39673-50-2 | 15.00% | 6.00 g |
| Vanilla FK3685 | 5.00% | 2.00 g |

After the individual flavor combinations were added to the batches, each batch was mixed on high agitation for about 10 minutes. Then each batch was mixed on low agitation (setting 2) for 5 minutes. The mixer was switched to first gear and each batch is mixed on setting 2 until ready to use.

Once the flavored batches of film-forming solution were prepared, a porous substrate was embedded with the water-soluble film as follows:
1. A 6″ wide filter paper, a tea bag like material which is the porous substrate to be embedded, was wetted with deionized water or 0.5% Tween solution.
2. The filter paper was placed on lop of a supporting layer of HDPE paper (6″ wide) and taped at one end. The layered substrates (filter paper and supporting layer) were then clamped onto the K-Coater.
3. 40 g of film-forming solution was dragon on the filter paper.
4. The filter paper was then dried at approximately 80-85° C. for approximately 13-20 minutes until the film-forming solution formed a film embedded in the filler paper.
5. After the film dried, the film-embedded filter paper substrate was removed from the supporting layer for further use.
6. The film-embedded filter paper was folded over itself to form a pouch having a closed volume. The film-embedded filter paper was then heat sealed using a Van der Stahl Fuji Impulse heat sealer. The film-embedded filter paper heat sealed well.

### Example 2

The method in Example 1 was repeated, except that polyethylene terephthalate (PET) (commercially available as untreated Mylar® (DuPont)) was utilized as the supporting layer. Similar results were achieved.

### Example 3

A film-embedded substrate was made as set forth in Example 2, except the filter paper was laminated to the PET supporting layer by applying heat. This allowed the filter paper to move at the same rate as the PET layer. Applying water to the substrate prior to lamination minimized wrinkling and creasing.

### Example 4

Different flavor batches were used in this example. The flavor combination added to the first batch was cherry flavor sweetened with the polyol Xylidex® (Cargill). The flavor combination added to the second batch was two types of wintergreen flavor. The flavor added to the third batch was a citrus flavor.

Film-embedded substrates were made as set forth in Example 1, except the 6″ wide filter paper was reduced to a size that allowed the paper to be placed at a minimum of 0.25″ away from both of the side dams of a K-Coater. This reduced creasing and bunching of the filter paper. Flow properties appeared to be the same along either axis of the diamond pattern in the filter paper.

### Example 5

Film-embedded substrates were formed as set forth in Example 1, except different flavor batches were used. The first flavor batch included a citrus flavor. The second batch included mint and menthol. The third batch included orange and orange cognac flavors. The fourth batch included cinnamon and peppermint flavors. Slight mottling was observed with the citrus flavor in the first batch. Some cracking in the film was seen with the cinnamon flavor in the fourth batch. The mint/menthol and orange cognac flavors in batches two and three, respectively, showed good results.

### Example 6

Film-embedded substrates were made as set forth in Example 2, except an approximately 12″ wide filter paper was placed on the supporting layer (PET). The filter paper substrate and PET supporting layer were held together through static charges. The method provided good results and a smooth film.

### Example 7

Film-embedded substrates were made as set forth in Example 2, except the filter paper was laminated to the supporting layer (PET) by the use of water applied to the filter paper. This experiment was conducted on a 30″ film coating line. The temperature of the drying process for the three ovens was 80-120 C with fan speed of 80-100% and humidity between 35%-65%. The line speed was maintained between 1 m/min-8 m/min, preferably 3 m/min The roller tensions were held between 200-300N. The method provided good results and a smooth film.

What is claimed is:

1. A pouch for administering an active component, comprising:
at least one porous semi-permeable substrate; and
at least one water-soluble film at least partially embedded in said at least one porous substrate said film embedded substrate encompassing a closed volume.

2. The pouch of claim 1, wherein said at least one porous semi-permeable substrate comprises a water-insoluble material.

3. The pouch of claim 2, wherein said water-insoluble material is selected from the group consisting of fiber, paper, water-insoluble polymers, cloth and fabric.

4. The pouch of claim 1, wherein said water-soluble film comprises at least one water-soluble polymer.

5. The pouch of claim 4, wherein said water-soluble polymer is capable of heat-sealing.

6. The pouch of claim 4, wherein said water-soluble polymer is selected from the group consisting of hydroxypropyl methylcellulose, polyethylene oxide, polyvinyl alcohol and combinations thereof.

7. The pouch of claim 6, further comprising polydextrose.

8. The pouch of claim 1, wherein said water-soluble film comprises at least one active component.

9. The pouch of claim 8, wherein said active component is selected from the group consisting of food products; botanicals; herbals; minerals; insects; nutraceuticals; pharmaceutical agents; cosmetic agents; drugs; medicaments; antidotes; vaccines; antigens or allergens; mouthwash components; flavors; fragrances; enzymes; preservatives; sweetening agents; colorants; spices; vitamins; and combinations thereof.

10. The pouch of claim 9, wherein said colorant comprises a whitening agent.

11. The pouch of claim 1, wherein said water-soluble film has a dissolution rate of about 1 minute to about 2 minutes.

12. The pouch of claim 1, wherein said water-soluble film has a dissolution rate of about 30 minutes to about 60 minutes.

13. The pouch of claim 1, wherein said water-soluble film has a dissolution rate of up to about 24 hours.

14. The pouch of claim 1, further comprising an active component contained in said closed volume.

15. The pouch of claim 14, wherein said active component is selected from the group consisting of: food products; botanicals; herbals; minerals; insects; nutraceuticals; pharmaceutical agents; cosmetic agents; drugs; medicaments; antidotes; vaccines; antigens or allergens; mouthwash components; flavors; fragrances; enzymes; preservatives; sweetening agents; colorants; spices; vitamins; and combinations thereof.

16. The pouch of claim 1, further comprising at least one tobacco product contained in said closed volume.

17. The pouch of claim 1, wherein first and second porous substrates are provided, wherein said first porous substrate comprises a sheet-like member and said second porous substrate comprises a sheet-like member, said first and second porous substrates being in perimetric face-to-face engagement with one another.

18. The pouch or claim 17, wherein said first porous substrate and said second porous substrate are fused along at least a portion of said perimetric face-to-face engagement.

19. The pouch of claim 1, wherein one substrate is provided, said substrate being folded to define said closed volume.

20. The pouch of claim 1, wherein said water-soluble film has a thickness of about 20 micron to about 1000 micron.

21. The pouch of claim 1, wherein said water-soluble film comprises an anti-foaming agent.

22. The pouch of claim 1 wherein said water-soluble film comprises a flavor present in amounts of about 5% to about 27% by weight of said film.

23. The pouch of claim 22, wherein said water-soluble film further comprises an emulsification system, said emulsification system comprising propylene glycol alginate, polyoxyethylene sorbitan monooleate and sorbitan monooleate.

24. The pouch of claim 1, wherein said water-soluble film is extruded.

25. The pouch of claim 1, wherein said water-soluble film further comprises an ionic component that imparts or maintains a charged environment to the water-soluble film.

26. A method of making a pouch for administering an active component, comprising the steps of:
(a) providing a water-insoluble porous semi-permeable substrate;
(b) at least partially embedding the porous substrate with a water-soluble film; and
(c) folding the at least partially embedded porous substrate to define a closed volume.

27. The method of claim 26, further comprising the step of heat-sealing the at least partially embedded porous substrate to itself.

28. A method of making a pouch for administering an active component, comprising the steps of:
(a) providing a water-insoluble porous semi-permeable substrate;
(b) at least partially embedding the porous substrate with a water-soluble film to form a first and a second sheet-like film embedded substrate;
(c) positioning said first and second embedded substrates in perimetric face-to-face engagement with one another; and
(d) fusing said first and second embedded substrates along at least a portion of said perimetric face-to-face engagement.

29. A method of delivering multiple active components into a body cavity of an individual, comprising the steps of:
(a) providing a pouch comprising:
(i) at least one porous semi-permeable substrate encompassing a closed volume;
(ii) at least one water-soluble film at least partially embedded in the at least one porous substrate, said water-soluble film comprising a first active component; and
(iii) a second active component contained in the closed volume;
(b) applying the pouch into the body cavity of the individual, and
(c) allowing the at least one water-soluble film to dissolve and release the first active component into the body cavity of the individual in combination with the second active component.

30. The method or claim 29, wherein the body cavity is an oral cavity, and wherein said first active component comprises a flavor and said second active component is selected from the group consisting of food products, pharmaceutical agents, nutraceuticals and cosmetic agents.

31. A method of delivering an active component in combination with a tobacco product into an oral cavity of an individual, comprising the steps of:
(a) providing a pouch comprising:
(i) at least one porous semi-permeable substrate encompassing a closed volume;
(ii) at least one water-soluble film at least partially embedded in the at least one porous substrate, said water-soluble film comprising an active component; and
(iii) a tobacco product contained in the closed volume;
(b) applying the pouch into the oral cavity of the individual; and
(c) allowing the at least one water-soluble film to dissolve and release the active component into the oral cavity of the individual in combination with the tobacco product.

32. An herbal product comprising:
(a) at least one porous semi-permeable substrate;
(b) at least one water-soluble film at least partially embedded in the at least one substrate, said film embedded substrate encompassing a closed volume;
(c) an herbal component in said closed volume.

33. The method of claim 32, wherein said herbal component is selected from the group consisting of: agrimony, alfalfa, aloe, angelica, anise, arjuna, arnica, Artemisia, ashwagandha; astragalus, avena, barberry, bayberry bilberry, bdellium gum, bilberry, birch, bissy nut, bitter orange, black cohosh, black currant oil, black walnut, blessed thistle, blue cohosh, blue vervain, borage, burdock, burdock, butcher's broom, calendula, cascara sagrada, catnip, cat's claw, cayenne, celery seed, chamomile, chaparral, chickweed, chrysanthemum, cinnamon, cleavers, clove, comfrey, coptis, cordyceps, cranberry, cyani flowers, damiana, dan shen, dandelion, devil's claw, dong quai, Echinacea, elderberry, elecampane, ephedra, eucalyptus, eyebright, false unicorn, fennel seed, fenugreek, feverfew, flax seed oil, garcinia cambogia, garlic, gentian, ginger, gingko, ginseng, goldenseal, gotu kola, hawthorn, holy basil, ho she wu, hops, horehound root, horseradish, horsetail, hydrangea, hyssop, irish moss, juniper berry, kava, kelp, khella, lady slipper, lamb's quarter, lavender, lemon balm, licorice, lobelia, male fern, mandrake, marshmallow root, mate, medical marijuana, milk thistle, morinda, motherwort, mullein, myrrh, nasturtium, neem oil, noni, oatstraw, olive leaf, oregano oil, Oregon grape root, pan pien pien, papaya, parruva brava, parsley, passionflower, pau d'arco, peppermint, periwinkle, pippali fruit, poke, prickly ash, psyllium, queen of the meadow, quercetin, raspberry leaf, red clover, reishi, rhubarb, rooibos, rosemary, safflower, sage, sarsaparilla, saw palmetto, schisandra, senega root, senna, skullcap, sheep sorrel, shepherd's purse, shiitake, Siberian ginseng, slippery elm, spirulina, squaw vine, St. John's wort, stinging nettle, suma, tea tree oil, thyme, turkey rhubarb, turmeric, usnea, uva ursi, valerian, vitex, watermelon seeds, white oak, white willow, wild cherry bark, wild yam, willow bark, wood betony, wormwood, yarrow, yellow dock, yerba santa; and combinations thereof.

34. The method of claim 32, wherein said water-soluble film further comprises an active.

35. The method of claim 34, wherein said active is a flavor agent.
